(19) United States
(12) Patent Application Publication
(10) Pub. No.: US 2025/0149130 A1
(43) Pub. Date: May 8, 2025

(54) Noteless Electronic Medical Records System

(71) Applicants: Alex Butler, West Roxbury, MA (US); Jacob Kantrowitz, Boston, MA (US); Jackson Steinkamp, Warwick, RI (US); Abhinav Shama, Vancouver (CA)

(72) Inventors: Alex Butler, West Roxbury, MA (US); Jacob Kantrowitz, Boston, MA (US); Jackson Steinkamp, Warwick, RI (US); Abhinav Shama, Vancouver (CA)

(21) Appl. No.: 18/387,911

(22) Filed: Nov. 8, 2023

Related U.S. Application Data
(60) Provisional application No. 63/423,842, filed on Nov. 9, 2022.

(51) Int. Cl. G16H 10/60

(52) U.S. Cl. CPC G16H 10/60

(57) ABSTRACT

A method for noteless electronic medical records system (NEMRS) is disclosed. NEMRS is system that does not require health care professionals to take hand written notes, but rather, treats a patient's medical chart as a single, dynamically updating, and fully collaborative workspace. All information is organized by clinical topic or problem on a set of dynamic, ‘digital cards.’ Version history functionality is used to enable granular tracking of changes over time based on a template engine. The centralized system is highly customizable to individual workflows and enables each individual user to decide which data should be structured and which should be unstructured. An object of the invention is to allow health care professionals to leverage the advantages of structured-templating and clinical decision support as desired without any programming knowledge. The system is designed to facilitate real-time, fully collaborative documentation and communication among multiple clinicians and minimize redundant note taking.

### CROSS-REFERENCE TO RELATED APPLICATIONS

The present application includes subject matter disclosed in and claims priority to a provisional application entitled “Noteless Electronic Medical Records System” filed Nov. 9, 2022 and assigned Application No. 63/423,842 describing an invention made by the present inventor.

### FIELD OF THE INVENTION

The present invention generally relates to medical records. More specifically, it relates to an easy-to-use, medical records system based on dynamic cards.

### BACKGROUND

Clinicians spend much of their workday using electronic medical records (EMRs), both getting data in—entering new information about patients—and getting data out—searching for and summarizing past information. Some studies estimate that ambulatory physicians spend approximately 50% of their work time navigating, searching, and entering information into the EMR. Therefore, optimizing clinical information management systems is crucial for clinician efficiency, satisfaction, and high-quality patient care. Poorly designed information management systems lead to the proliferation of out-of-date or incorrect information, increased time spent searching medical charts, medical errors, and clinician burnout, while limiting the effective use of EMR data for individual or population-level applications. Given the scope and impact of the problem, we must critically evaluate the core conceptual assumptions that underpin our current practices. For instance, are notes the best organizational unit for unstructured clinical data? Should clinicians in different teams always document in separate locations? Which data should be recorded and stored in a structured fashion, and which in an unstructured fashion? Historically, these questions have been informed less by principled usability concerns and more by regulatory requirements, vendor incentives, and holdover intuitions from the era of paper records, which have fundamentally different capabilities and limitations than electronic systems. A multitude of electronic medical records systems have evolved over the years to streamline medical record keeping for doctors and health care professionals. U.S. Pat. No. 5,924,074A disclosed a medical records system; however, it proposed a system designed largely as a patient data repository. United States patent No. US20150261917A1 disclosed a novel method of collaborative medical data entry; however, it does not focus on a new means to streamline medical data entry in the field. United States patent No. US20130191161A1 disclosed a clinical decision interface; however, it is also not focused on streamlining the data processes in the field. U.S. Pat. No. 10,037,820B2 disclosed a contextual display of medical information for health care professionals; however, it does not include other types of data (lab results, imaging) and is not organized by medical problem with cards instead relying on a graphical user interface (GUI) for all information. What is needed is a dynamic medical records system that minimizes data entry times for doctors working in the field.

### SUMMARY OF THE INVENTION

The device herein disclosed and described provides a solution to the shortcomings in the prior art through the disclosure of a NEMRS. A main object of the invention is to streamline interactions between a medical professional and the medical records entry and query process. This enhancement is achieved through a system designed around small-scale changes of individual data elements without requiring redocumentation of the remainder of the unchanged data during each visit.

Another object of the invention is to reduce information overload, information scatter, and error propagation. The NEMRS dissolves the sharp boundary between notes written by different teams in favor of a default collaborative workspace. For example, when logged into the system all stakeholders can view patient information and records by multiple health care specialists to minimize redundant, time-consuming entries.

Another object of the invention is to allow medical records to be presented as guides and in some cases a teaching tool to facilitate learning among health care professionals. For instance, if a clinician indicates that a patient has congestive heart failure, the system should present the clinician with a framework for how to think about that problem, including relevant data to be collected, active diagnostic possibilities, actions to be considered (including diagnostic tests, therapeutic orders, consults, and dispositional concerns), and links to external information resources. These frameworks are informed by other information already captured in the chart—for instance, if a cardiac history was already captured in the context of a coronary artery disease workup, that information should be viewable by default from within the CHF problem, removing the need to search for that information (and, if unchanged, the need to redocument it).

It is briefly noted that upon reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods, and systems for carrying out the several purposes of the present disclosed device. It is important, therefore, that the claims be regarded as including such equivalent construction and methodology insofar as they do not depart from the spirit and scope of the present invention. As used in the claims to describe the various: inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they affect the activity or action of the listed elements. The objects features, and advantages of the present invention, as well as the advantages thereof over existing prior art, which will become apparent from the description to follow, are accomplished by the improvements described in this specification and hereinafter described in the following detailed description which fully discloses the invention but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 is a diagram showing the organization of the major conceptual entities in the NEMRS.

FIG. 2 shows examples of NEMRS card templates.

FIG. 3 shows structured data display operators for NEMRS card fields.

FIG. 4 shows a diagram of the overall organization of a NEMRS patient interface.

FIG. 5 shows a diagram of another NEMRS patient interface organization.

FIG. 6 shows a diagram of data flows between the network and stakeholders.

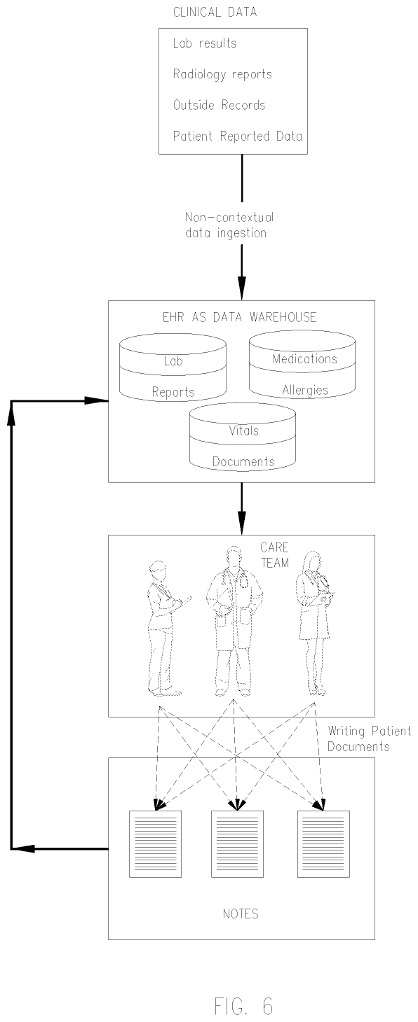

Other aspects of the present invention shall be more readily understood when considered in conjunction with the accompanying drawings, and the following detailed description, neither of which should be considered limiting.

### DETAILED DESCRIPTION OF FIGURES

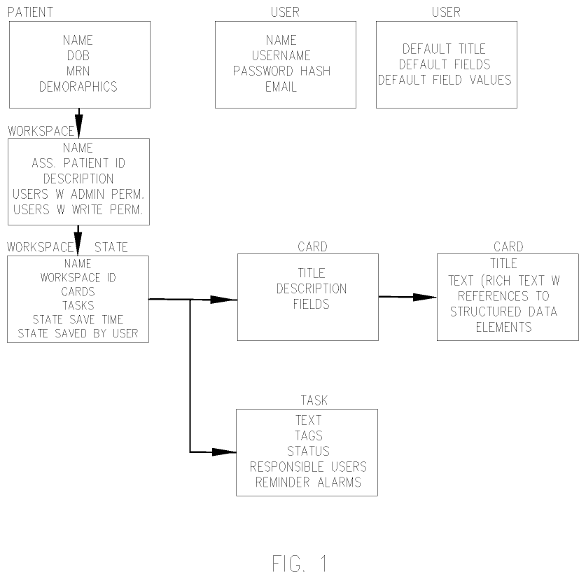

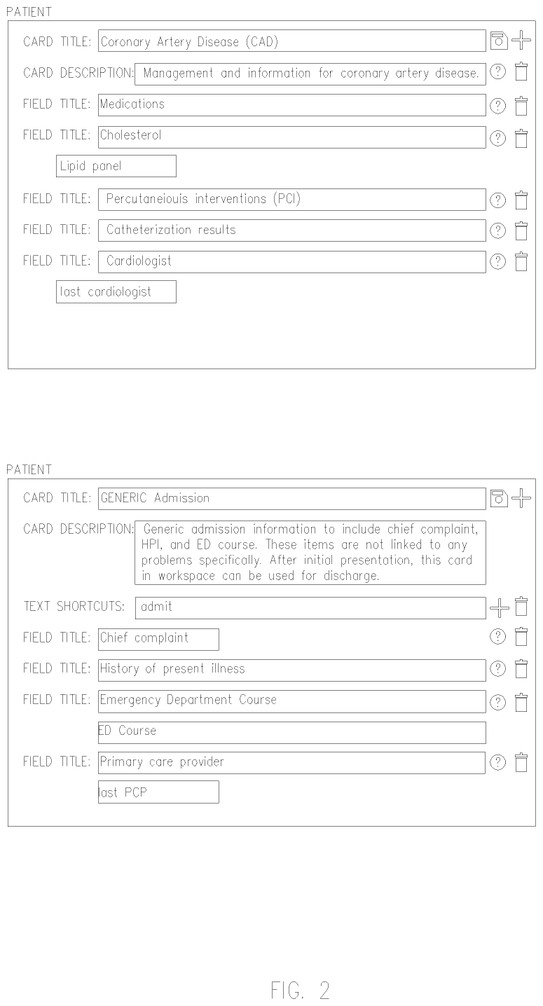

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure. FIG. 1 shows the preferred embodiment of the NEMRS' major components within the software along with their implementation. This software is run on a server with one or more processors and one or more mobile or computer-readable non-transitory storage media coupled to one or more of the processors and comprising instructions operable when executed by one or more of the processors. The software consists of one or more computer-readable non-transitory storage media comprising software that is operable when executed by the server to and based on data stored by the processor and software as a service to streamline interactions between medical professionals and medical records. Each of these key features is designed to decrease information chaos and redundancy—particularly the duplication and scatter of related information in disparate locations. The figure shows the overall relationships between the different conceptual entities and data structures in the MongoDB database. FIG. 2 shows examples of NEMRS card templates. Under traditional, note-based paradigm medical record keeping, all information covering a particular time slice of a patient's medical life (e.g., a single day of hospitalization) must be stored together in one note, regardless of content. As discussed above, this organizational scheme forces users to write notes that are overloaded with information or that are underloaded, with information scattered across many notes. Instead of the note as time slice approach, the NEMRS takes inspiration from work on the problem-oriented medical record and organizes information by topic to facilitate charts that teach and guide. In the NEMRS software, each medical topic is represented by a ‘digital card,’ which holds a set of information related to a patient's medical history along with an associated medical topic that is selected by a health care professional and is assigned to a patient's record. Each digital card consists of a title and multiple fields that provide additional structure within the topic as shown in FIG. 2. These digital cards are frequently used to capture and summarize individual medical problems; for instance, a heart exacerbation digital card might hold fields representing the patient's history of present illness, relevant physical exam findings, outpatient clinicians, laboratory and imaging results, and treatment orders. Over time, as the history of a patient's medical condition or topic “changes, as does the associated card. As an example, a card for “congestive heart failure” may collect and store relevant information on day of diagnosis and track relevant imaging findings or medication changes throughout a patient's. This allows for rapid appreciation of a single disease course while providing a dedicated area for relevant information storage and retrieval. Additionally, as the current healthcare system requires notes at key points of care transition for billing and hospital administration (e.g., admission to the hospital, visit to the ED), these ‘digital cards’ can be also be used as note templates for the process of data collection and sharing. Though these cards may not be considered in a given patient's list of medical conditions in the future, they still serve: an important purpose in the current healthcare landscape. A sample of this use of card is included in the bottom half of this figure.

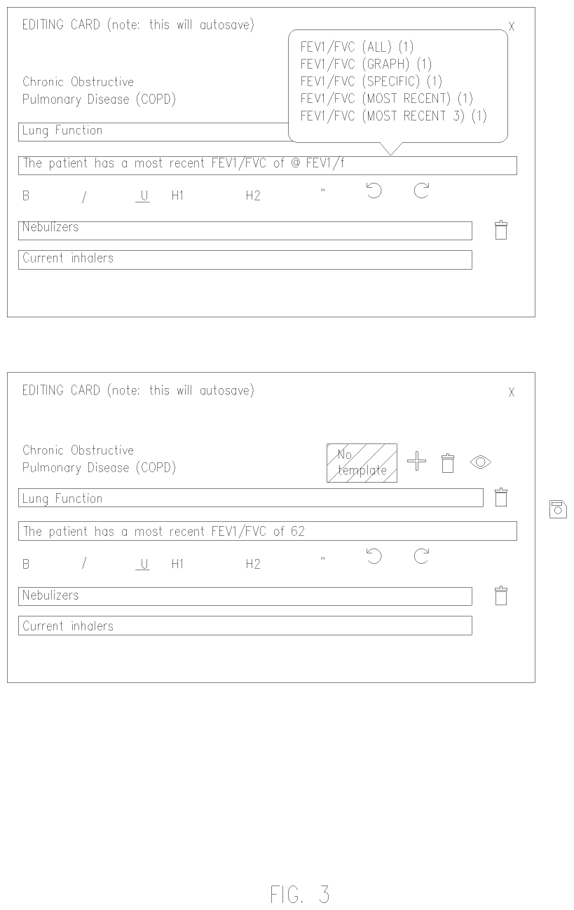

FIG. 3 shows structured data display operators for NEMRS card fields. NEMRS digital cards are designed to be flexible enough to capture data relevant to any givenmedial topic, such as “demographic data”, “cardiology consult recommendations”, “list of outpatient clinicians”, “nursing comments”, or “overnight updates.” This feature enables institutions, teams, or individual clinicians to develop digital cards customized to their own individual workflows. Many of the fields are, by default, free-text and retain all the advantages of textual data while adding flexible structure. Some fields, including the ‘Nebulizer’ field in the included image, can be linked to structured data within the EMR to display real-time relevant information, such as active nebulized medications.

Each digital card field's data are stored and updated separately in the database rather than every topic being stored together in a single block of free text. This function enables ‘granular viewing’ of individual cards or fields and their changes over time, in the case where a clinician wants to quickly see the history of a patient's illness. However, the default chart view shows all currently active cards for a patient regardless of how recently each piece of information has changed. This is sensible, as different medical information update at different frequencies. Even for the same problem, a patient's acute status may change daily, whereas their outpatient medications may change monthly, their providers change still less often, and their demographics may never change. Therefore, clinicians can get a quick understanding of the current state of the patient without having to navigate back through numerous notes to find updates that occurred at different times. The NEMRS shares these advantages with other forms of problem-based or topic-based charting. By using digital cards as the basic organizational principle, we escape the trade-off between information overload and information scatter.

NEMRS facilitates granular updates to individual topics without requiring large-scale redocumentation and information overload. For instance, if everything about a patient's illness has remained the same from day one to day two, the digital card does not need to be modified at all. Instead, the responsible clinician simply attests that no information has changed, which saves time and does not create a duplicate copy of the same data (as is the case with notes). Updates that consist of only one or two factual updates to the chart (i.e., a telephone call to refill medication) require clinicians to only update a single card field. Unlike a note-based organization, which would scatter topically related information across multiple notes, the relevant data will be stored together regardless of when it was documented, enabling easier viewing of longitudinal changes. Therefore, the digital card-based system cuts down on both information overload and information scatter. This structure also enables readers (or automated systems) to quickly identify which cards and fields have changed from update to update and which have remained the same. We will discuss this further in the Version History section.

The NEMRS digital cards also facilitate real-time collaboration. Multiple clinicians work on individual cards or even different fields within the same card at the same time, reducing the incentive for individuals on the same team to create separate notes. Furthermore, digital card-based organization can reduce the number of separate clinical threads; ideally, inpatient consultants would be responsible for individual cards or fields within a general inpatient workspace, obviating the need for the consultant to redocument large amounts of the patient's history of present illness medication list, etc. Real-time collaboration is facilitated using web sockets, which allow for two-way communication between client and server; when a client makes any update to a card within a patient workspace, the server will push the update via the web socket, enabling other users to see the changes immediately. This facilitates the reconceptualization of the chart as a dynamic living workspace.

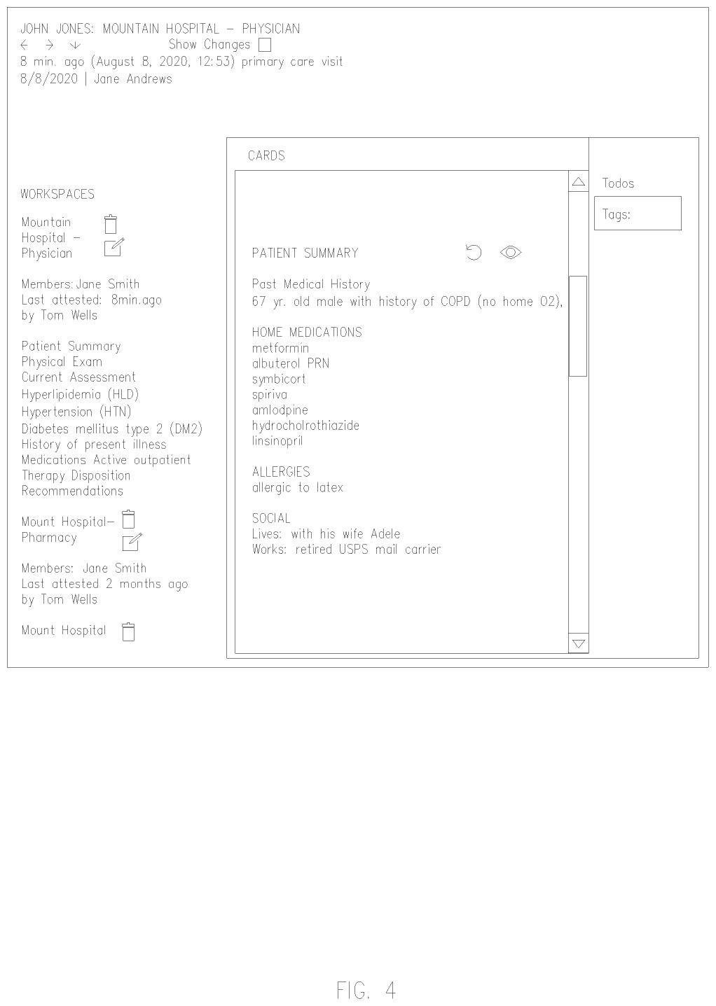

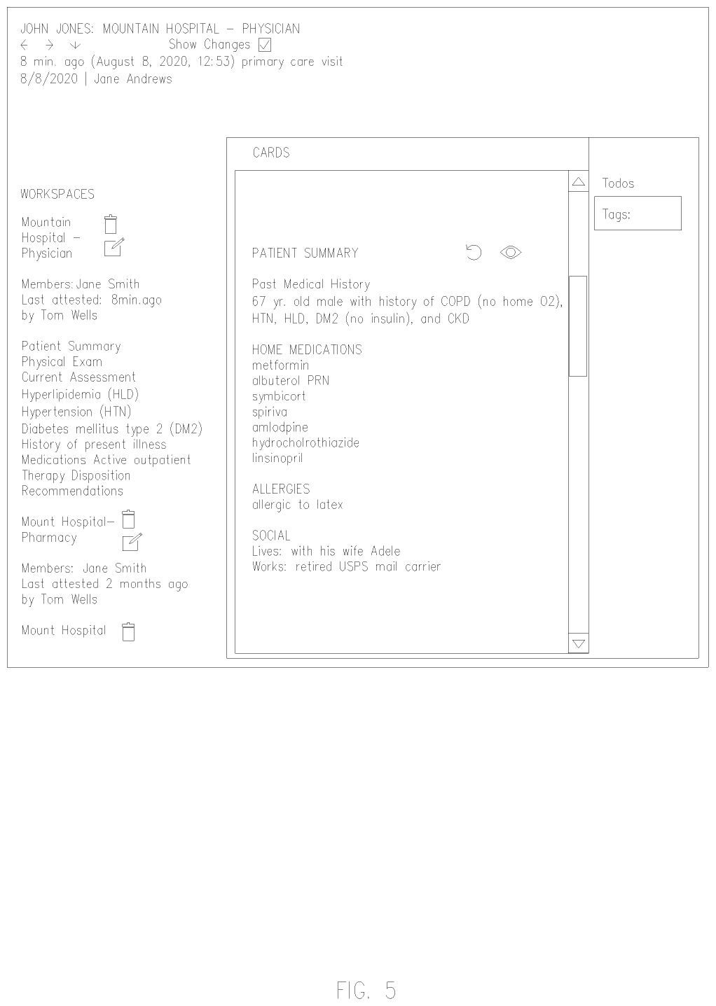

FIG. 4 shows a diagram of the overall organization of a NEMRS patient interface known as a ‘structured data display operator.’ Structured data elements are pulled into unstructured documentation using an @ symbol operator inside any of the free text card fields, akin to a mention or hashtag system in other software as observed in FIG. 4. For instance, if “@sodium” is typed inside a card field, a set of options related to the stored sodium laboratory values will appear. In its simplest form, a reference to the latest test result will be pulled into the text field in real time as displayed in FIG. 5. Critically, this structured data value is not a disjoint piece of text but a reference that can be updated as new information arrives. If the user wants to track the latest value of sodium, and a new value of sodium is input into the system, the textual reference will display a refresh icon, suggesting that the displayed value is no longer the latest value. The user can choose whether they wish to make use of the refresh icon to update their textual documentation.

This system is designed to prevent the propagation of incorrect values because when a clinician forgets to update the text—a scenario that has the potential for significant medical error. NEMRS allows each structured data element to be displayed in multiple formats—a “last value” option, an “all values” option, a “specific value” option (which does not change with time), and a graph option, which enables a real-time updating graph of the latest values of a particular structured data element, such as a laboratory value. These data elements are situated within the free text card fields and removes the requirement to navigate with multiple clicks to separate graph viewing interfaces and showing topic-relevant information in the same place—preventing information scatter, underload, and lost time. To facilitate flexible, customizable workflows, NEMRS takes the widest possible view of what counts as structured data by enabling individual users to define and create their own structured data elements, as well as enter any structured data value themselves (e.g., laboratory values obtained from outside organizations). This includes traditional structured data elements, such as laboratory results, but is also designed to work with other numeric values (ejection fraction, pack-years of tobacco use, and risk scores) in addition to string values and categorical values (code status and the patient's current outpatient pulmonologist). This feature empowers individual clinicians to quickly improve their templating systems.

In the NEMRS, digital cards are used as part of a ‘template engine’ that creates a granular templating system consisting of a set of prespecified fields with optional prespecified default text in each field. Inside a patient's workspace, digital cards can be quickly instantiated from the templates. This default text can include structured data elements, enabling the clinician to (for instance) auto-populate a reference to the latest ejection fraction when a digital card is instantiated from a template. Trainees, in particular, may benefit from the template engine, which provides a framework for thinking about a: particular medical topic, including important diagnostic considerations, therapeutic decision points, and signs of patient status change, all of which can be included within a card template. Physicians play multiple roles, and these teaching medical charts can help us to be better clinicians, educators, and learners by providing perpetually up-to-date resources naturally within our existing workflows. Digital card templates can include embedded text from external resources such as guideline organizations and research studies, enabling not only clinical refreshers but also ongoing medical education.

Although NEMRS is designed to cut down on the number of distinct clinical threads and the information overload that results from multiple clinicians or teams redocumenting the same information, there are potential use cases where clinicians should work in separate workspaces. Therefore, NEMRS allow for the creation of separate workspaces for the same patient, which recaptures the notion of separate threads. The system requires only a single click to navigate from one workspace to another in the same patient's chart. Each workspace has specific user permissions: clinicians with write permission to a workspace can see the most up-to-date version of a workspace, even if it has not yet been attested. On the other hand, those with read permissions can only see the most recent attested version to prevent acting on incomplete information. For custom workflows, digital cards from one workspace can be watched from another. Watching a digital card creates a read-only reference to the original card in the new workspace and enables a clinician to view relevant information documented by someone else from another workspace. When the original card updates, the watched copy of the card will also update in real time. This is meant to replace the current behavior under a note-based system in which clinicians copy-paste information (e.g., consult recommendations or a radiology report) from one text document into another to have all the information in one place. Although other design decisions of our system will ideally decrease the information scatter that motivates this behavior, card-watching.

Conversely, in many clinical care settings, relevant patient information is frequently passed between clinicians and staff through ancillary systems such as emails, pagers, SMS text messages, and separate handoffs. NEMRS includes a ‘task management’ or ‘to-do list tracking’ as a core feature representing real time collaboration. Each workspace includes a dynamically editable, fully collaborative to-do list that enables task management for that patient. To-do lists can be assigned to particular users, can be associated with reminders accompanied by push notifications, and can be given custom user tags to enable filtering and searching functionality. These to-do lists can be viewed in aggregate across a group of patients, enabling efficient inpatient team and outpatient panel management. Therefore, NEMRS includes a chat functionality as the primary mode of communication. The chat system enables direct messaging among clinical users in the system and includes a group chat functionality, which is similar to the chat functionality present in other medical organization systems. In addition, in NEMRS, each patient workspace is associated with its own additional chat, so short notes or messages related to a particular patient can be stored. However, unlike many other chat systems, ours is public and, thus, auditable. The public nature of the chat enables any system user, including physician colleagues or learners, nurses, and other staff, to understand the clinical decisions that have been made for their patients and to learn from these conversations, as opposed to these happening in closed-door sessions. Within the workspaces, we include a real-time cursor tracking feature: a user in a patient chart can see which other users are editing that workspace and where their cursor is located to prevent multiple users from editing the same information at the same time and facilitate real-time collaboration (e.g., by serving as a jumping-off point for a chat conversation between a primary clinician and a consultant).

Although some may be hesitant to treat the chart as a collaborative workspace, based on fears of medicolegal reprisal or insufficient assignment of responsibility to individuals, NEMRS is capable of handling the responsibility assignment issue at least as well as note-based systems. All digital card activity (addition or deletion of a card, editing of a card title or field text, etc.) is tracked in a separate logging table, enabling fine-grained assignment of responsibility for individual edits to particular users. These logs also enable the ascription of responsibility for each piece of text in a workspace to particular users and particular time stamps. This feature can be helpful not only in cases of ascribing responsibility but also when a user wishes to orient themselves to a new chart and understand what information has been recently updated and what needs verification or updating.

Another major source of information scatter rests at the level of the team or patient group. An extremely common medical records system uses case involves clinicians managing groups of patients (e.g., an inpatient team or an outpatient panel) and either (1) looking for updates in the state of any patient in the group or (2) running the list—identifying the status and action items of a group of patients in rapid sequence. Team management and list-running are other core features in NEMRS. The team view interface aggregates the latest state of all workspaces for all patients on a particular team, enabling highly granular actions to be taken for individual patients from a single screen without any requirement to navigate between charts. The team view displays the list of digital cards for each workspace, enabling quick addition or editing of specific card fields with new single pieces of information. This enables immediate placement of information in topic-appropriate locations—a feature designed to facilitate keeping the chart organized, preventing the need to redocument the same information in different places or scatter information across multiple locations. In addition, the to-do items for each workspace are aggregated and viewable on one page; enabling efficient list running even for large groups of patients.

In view of the disclosure provided herein, a mobile application is created by techniques known to those of skill in the art using hardware, languages, and development environments known to the art. Those of skill in the art will recognize that mobile applications are written in several languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof. The software also compatible with a plurality of operating systems such as, but not limited to: Windows™, Apple™, and Android™, and compatible with a multitude of hardware platforms such as, but not limited to: personal desktops, laptops, tablets, smartphones and the like. Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK. Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications. In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the scope of the invention.

What is claimed is:

1. A system for streamlining interactions between medical professionals and medical records entry and query processes comprising the following parts:
a) a server with one or more processors and one or more mobile or computer-readable non-transitory storage media coupled to one or more of the processors and comprising instructions operable when executed by one or more of the processors; and
b) one or more computer-readable non-transitory storage media comprising software that is operable when executed by the server to and based on data stored by the processor and software as a service for streamlining interactions between medical professionals and medical records.

2. The system of claim 1, wherein the software having digital cards containing patient medical history along with information relating to an associated medical topic for streamlining interactions between medical professionals and medical records.

3. The digital cards of claim 2, wherein the digital cards enabling institutions, teams, or individual clinicians to develop digital cards customized to their own individual workflows.

4. The digital cards of claim 2, wherein the digital cards being accessed by multiple clinicians in different fields at the same time reducing the incentive for note taking.

5. The digital cards of claim 2, wherein the digital cards being part of a template engine that creates a granular templating system consisting of a set of prespecified fields with optional prespecified default text in each field.

6. The digital cards of claim 2, wherein the digital cards include embedded text from external resources such as guideline organizations and research studies, enabling clinical refreshers and ongoing medical education for trainees.

7. The digital cards of claim 2, wherein all digital card activity being tracked in a separate logging table, enabling fine-grained assignment of responsibility for individual edits to particular users.

8. The system of claim 1, wherein the software having a workspace that includes to-do lists with reminders accompanied by push notifications and has permissions that enables clinicians to view relevant information documented by someone else from another workspace.

9. The logging table of claim 7, wherein the workspace having a real-time cursor tracking feature so a user can see others editing that workspace and where their cursor is located to prevent multiple users from editing the same information at the same time for facilitating real-time collaboration.

10. The system of claim 1, wherein the software having a patient interface allowing the patient to view test results in real time.

11. The system of claim 1, wherein the software allowing clinicians to view laboratory test results in multiple formats for preventing information scatter, underload, and lost time.

12. The system of claim 1, wherein the software having team management and list running functions interface that aggregates the latest state of all workspaces for all patients on a particular team, enabling highly granular actions to be taken for individual patients from a single screen without any requirement to navigate between charts.

13. The team management and list running functions of claim 12, wherein the interface having to-do items for each workspace aggregated and viewable for enabling efficient list running for large groups of patients.
